(19) United States
(12) Patent Application Publication
(10) Pub. No.: US 2015/0342882 A1
(43) Pub. Date: Dec. 3, 2015

(54) METHODS OF TREATMENT USING CADOTRIL COMPOSITIONS

(71) Applicant: Johnson & Johnson Consumer Inc., Skillman, NJ (US)

(72) Inventor: Der-Yang Lee, Flemington, NJ (US)

(21) Appl. No.: 14/821,072

(22) Filed: Aug. 7, 2015

Related U.S. Application Data
(63) Continuation-in-part of application No. 14/138,309, filed on Dec. 23, 2013.
Application No. 14/138,309 is a continuation-in-part of application No. 13/929,996, filed on Jun. 28, 2013.
(60) Provisional application No. 61/665,470, filed on Jun. 28, 2012.

(51) Int. Cl. A61K 9/107; A61K 31/265

(52) U.S. Cl. CPC A61K 9/1075; A61K 31/265

(57) ABSTRACT

Methods of treatment using cadotril compositions are disclosed.

### CROSS REFERENCE TO RELATED APPLICATIONS

This application is a continuation-in-part of U.S. applications Ser. No. 14/138,309; and claims the benefit of Ser. Nos. 14/138,309; 14/205,565; 13/929,996; 61,787,597; and 61/665,470, which are incorporated herein by reference.

### FIELD OF THE INVENTION

The present invention relates to methods of treatment using cadotril compositions.

### BACKGROUND OF THE INVENTION

Diarrhea or diarrhea is defined by the World Health Organization as a condition of having at least three loose or liquid bowel movements each day or as having more stool than is normal for that person.¹ It often lasts for a few days and can result in dehydration due to fluid loss. 1 See Diarrhoeal Disease Fact Sheet N° 330, World Health Organization, April 2013.

The most common cause of diarrhea is an infection of the intestines due to a virus, bacteria, or parasite, a condition known as gastroenteritis. These infections are often acquired from food or water that has been contaminated by stool, or directly from another person who is infected. A number of non-infectious causes may also result in diarrhea including: hyperthyroidism, lactose intolerance, inflammatory bowel disease, a number of medications, and irritable bowel syndrome, among others.

Prevention of infectious diarrhea is by improved sanitation, clean drinking water, and hand washing. Oral rehydration solution (ORS), which is clean water with modest amounts of salt and sugar, along with zinc tablets are often employed. In those with severe dehydration, intravenous fluids may be required.

About 1.7 to 5 billion cases of diarrhea occur per year.² It is most common in developing countries were young children get diarrhea on average three times a year. Worldwide, as of 2012, it is the second most common cause of death in children less than five (0.76 million or 11%).³ Frequent episodes of diarrhea are also a common cause of malnutrition and the most common cause in those less than five years of age. Other long term problems that can result include poor physical and intellectual development. 2See Diarrhoea: why children are still dying and what can be done, The United Nations Children's Fund, World Health Organization, 2009.3 See Diarrhoeal Disease Fact Sheet N° 330, World Health Organization, April 2013.

Chronic diarrhea can be the part of the presentations of a number of chronic medical conditions affecting the intestine. Common causes include ulcerative colitis, Crohn's disease, microscopic colitis, celiac disease, irritable bowel syndrome and bile acid malabsorption.

While antibiotics are beneficial in certain types of acute diarrhea, they are usually not used except in specific situations as some bacteria develop antibiotic resistance. Antibiotics themselves can also cause diarrhea, and antibiotic-associated diarrhea is the most common adverse effect associated with treatment using general antibiotics.

Anti-motility agents like loperamide are also effective at reducing the number of stools but not the duration of disease.⁴ 4 See Dupont, H. L., Acute infectious diarrhea in immunocompetent adults, New England Journal of Medicine, 2014, 370:1532-40.

Probiotics are “friendly” bacteria that have proven beneficial in the treatment of diarrhea.⁵ 5 See Allen S. J., et al., Probiotics for treating acute infectious diarrhea (Review), Cochrane Database of Systematic Reviews 2010, Issue 11. Art. No.: CD003048. DOI: 10.1002/14651858.CD003048.pub3.

Racecadotril (shown below), also known as acetorphan or (RS)-benzyl N-[3-(acetylthio)-2-benzylpropanoyl]glycinate, is an antidiarrheal drug which acts as a peripherally acting enkephalinase inhibitor. Unlike other medications used to treat diarrhea, which reduce intestinal motility, racecadotril has an antisecretory effect, i.e., it reduces the secretion of water and electrolytes into the intestine. Racecadotril exhibits an original intestinal antisecretory action, by protecting endogenous enkephalines against the degradation thereof By improving the biological activity of these neuropeptides at the delta opiate receptors, racecadotril reduces the net water and electrolyte efflux into the intestinal lumen, which flows are otherwise increased in diarrheal diseases of various origins. Racecadotril is selective in that the intestinal hypersecretion or reduced water and electrolyte absorption (which characterizes diarrhea and is responsible for severe states of dehydration) is greatly reduced without altering the transit.⁶ This model contributes to the particularly beneficial properties of racecadotril, as has been shown in clinical trials and post-marketing study.⁷ 6 Matheson A. J., et al., Drugs 2000, 59, 829; Schwartz J. C., Int. Antimicrob. Agents, 2000, 14, 81.7 Lecomte et al., Int. J. Antimicrob. Agents, 2000, 14, 81.

In clinical trials as well as in standard practice, racecadotril is generally administered in 100 mg capsules, taken three times a day, in order to ensure inhibition of the targeted enkephalinase throughout the day without interruption. A 175 mg twice a day (b.i.d.) tablet has also been studied. The t.i.d. dose for pediatric is 1.5 mg/kg resulting in a daily maximum of ≦6 mg/kg; for adult the t.i.d. dose is 100 mg resulting in a daily maximum of ≦400 mg.

Racecadotril is sold on the market in a number of countries under the tradename HIDRASEC® (trademark of SmithKline Beecham); TIORFAN® (trademark of Societe Civile de Recherche Bioprojet); TIORFAST® (marketed by Bioprojet Pharma); and TIORFIX® (marketed by Takeda). Marketed forms include dry granules filled into a hard gelatin capsule or a sachet; and tablets.

10 mg or 30 mg granules for oral suspension are available for pediatric use. The recommended dose is determined according to body weight: 1.5 mg/kg per dose (corresponding to 1 to 2 sachets). In infants less than 9 kg: the recommended dose is one 10 mg sachet three times daily; in infants from 9 kg to 13 kg: the recommended dose is two 10 mg sachet three times daily; and in children from 13 kg to 27 kg: the recommended dose is one 30 mg sachet three times daily; in children of more than 27 kg: the recommended dose is two 30 mg sachet three times daily

It is desirable to have additional formulations of racecadotril.

Dexecadotril (shown below), also known as R-acetorphan or N-[(R)-2-Benzyl-3-(acetylthio)propionyl]glycine benzyl ester; N—[(R)-2-[(Acetylthio)methyl]-1-oxo-3-phenylpropyl]glycine benzyl ester is the R enantiomer of racecadotril.

Ecadotril (shown below), also known as S-acetorphan or N—[(S)-2-[(Acetylthio)methyl]-1-oxo-3-phenylpropyl]glycine benzyl ester is the S enantiomer of racecadotril.

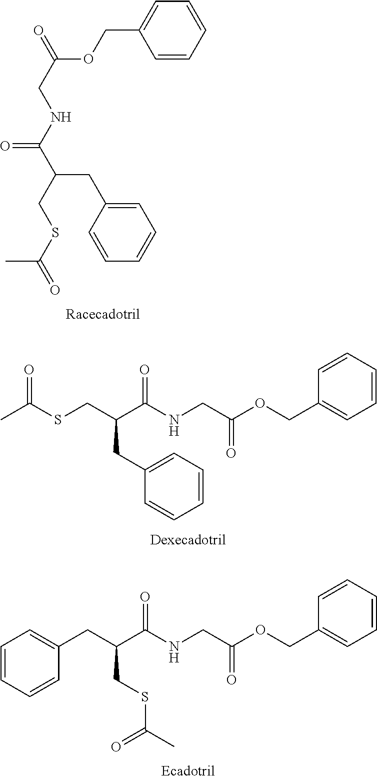 [SMILES: CC(=O)SCC(Cc1ccccc1)C(=O)NCC(=O)OCc1ccccc1 ; CC(=O)SC[C@H](Cc1ccccc1)C(=O)NCC(=O)OCc1ccccc1 ; CC(=O)SC[C@@H](Cc1ccccc1)C(=O)NCC(=O)OCc1ccccc1]

Throughout the disclosure “cadotril” will be used to include racecadotril, dexecadotril and/or ecadotril.

Racecadotril is a class II drug (as per Biopharmaceutical Classification System) with poor aqueous solubility and dissolution rate limited absorption. Racecadotril undergoes hydrolysis when it comes into contact with water. There are two major pairs of hydrolysis products, i.e., benzyl alcohol and EP Impurity C and ethanethioic acid (thioacetic acid) and EP Impurity G. Thiorphan, which is a product of the hydrolysis reaction, is not a major degradation product. Thiorphan (shown below) is the active metabolite of racecadotril, which exerts the bulk of its inhibitory actions on enkephalinase.

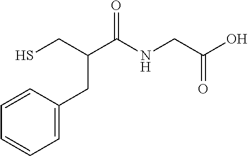 [SMILES: O=C(O)CNC(=O)C(CS)Cc1ccccc1]

Racecadotril is rapidly absorbed and entirely converted to thiorphan upon oral administration. The location of action is the epithelial cells of the mucosa of the bowel.

Known degradants of racecadotril are shown below.

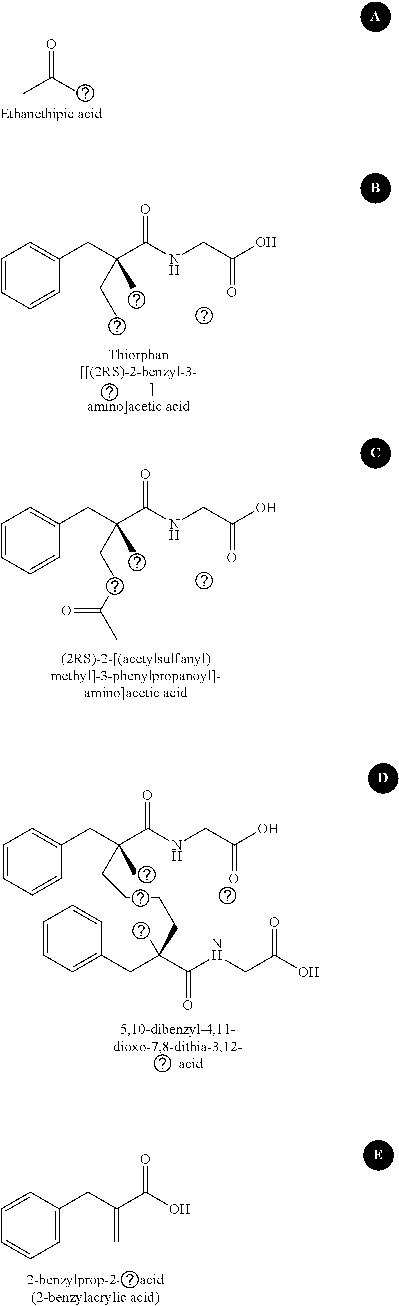

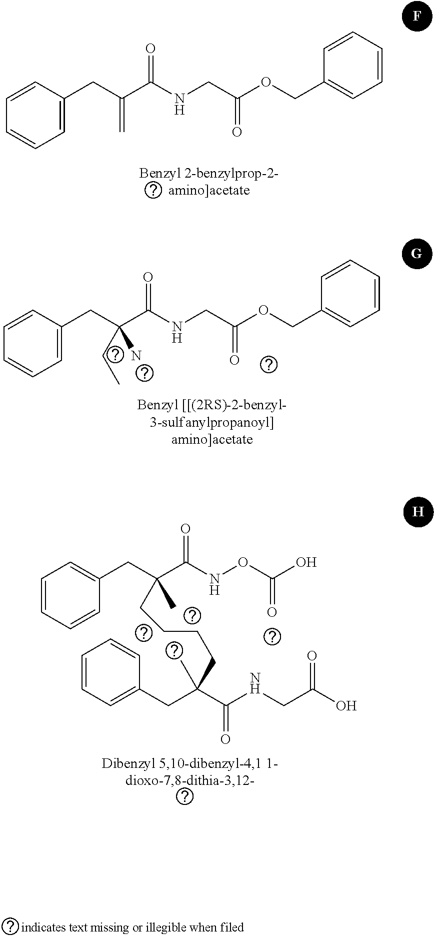

### European Pharmacopoeia 6.3, pg. 4283-4284.

U.S. Pat. No. 4,513,009 to Bioprojet discloses racecadotril and some of its therapeutic applications.

U.S. Pat. Nos. 5,331,008; 5,296,509; 5,208,255; and 5,136,076 to Bioprojet disclose enantiomeric forms of racecadotril.

U.S. Pat. No. 6,919,093 to Bioprojet discloses a dry powder racecadotril formulation that comprises coated granules and specified excipients.

U.S. Pat. No. 8,222,294 to Bioprojet discloses a combination that comprises racecadotril or dexecadotril with ondansetron or granisetron.

U.S. Pat. No. 8,318,203 to Bioprojet discloses a racecadotril tablet that comprises a coated core and specified excipients.

U.S. Application No. 20130331423 to Bioprojet discloses an aqueous suspension that comprises racecadotril.

WO2001097803 to GlaxoSmithKline discloses a granulate formulation comprising racecadotril and specified excipients.

U.S. Application No. 20020028248 to Tsukada et al. (now abandoned) discloses rapid-release microdispersible preparation containing ecadotril.

CN102133186 to Hainan Meida Pharmaceutical Co. discloses a liposome racecadotril solid preparation that comprises specified ingredients in specified relative weight ratios.

CN101103960 to Hainan Shengke Life Scientific Research Institute and CN102327234 to Hainan Honz Pharmaceutical Co., Ltd. each disclose racecadotril containing dry suspensions that comprises specified ingredients.

CN101264065 to Yancheng Suhai Pharmaceutical Co., Ltd. discloses a racecadotril dropping pill that comprises specified ingredients in specified weight ratios.

IN20110127511, IN20110127411 and IN201101191211 to Akums disclose pharmaceutical formulations that comprise (1) racecadotril and (2) ofloxacin and/or ornidazole.

IN20080088413 to Torrent Pharmaceuticals Limited discloses a resinate complex that comprises racecadotril.

IN20060165213 to Torrent Pharmaceuticals Limited discloses a taste-masked composition that comprises particles comprising racecadotril and a low melting excipient. The composition is prepared by dispersing racecadotril in a melt; cooling the dispersion at room temperature to form a solidified mass; and milling the solidified mass to obtain racecadotril particles.

U.S. Applications Nos. 20140005262; 20140275246; and 20140271832 to McNeil-PPC, Inc. disclose compositions that comprise racecadotril, at least one surfactant and a lipid.

U.S. Applications Nos. 20140005261; 20140274948; and 20140271831 to McNeil-PPC, Inc. disclose liquid compositions that comprise racecadotril and cyclodextrin.

U.S. Application No. 60/069,906, filed Oct. 29, 2014, to McNeil-PPC, Inc. discloses a method of manufacturing cadotril particles, comprising: melting a cadotril and a wax while mixing; dispersing the molten cadotril/wax mixture in hot water; transferring the hot cadotril/wax/water dispersion into another container containing cold water, wherein the dispersed droplets of cadotril /wax congeal and form fine/spherical particles; and filtering and drying the fine/spherical particles.

EP2749270 discloses a dispersible tablet comprising racecadotril coated with an acrylic acid polymer or a cellulose polymer by a wet granulation method.

CN102018707 discloses racecadotril and berberine HCl containing formulations. The reference discloses that soft gelatin capsules may contain glycerin and that suppository formulations may contain oils and surfactants.

Cannon, American Pharmaceutical Review, May (2011), discloses the use of Self-Microemulsifying Drug Delivery Systems (SMEDDS) or Self-Emulsifying Drug Delivery Systems (SEDDS) in pharmaceutical formulations.

Laddha et al., Brazilian Journal of Pharmaceutical Sciences (Impresso), Vol 50, No. 1 (2014), discloses an investigation into a self-microemulsifying drug delivery system (SMEDDS) to improve the in vitro dissolution of domperidone.

Zargar-Shoshtari et al., Chem. Pharm. Bull. 58(10) 1332-1338 (2010), discloses the investigation of Imwitor 308 SMEDDS as potential transdermal delivery systems for progesterone.

Mukherjee et al., JP 2010, 62: 1112-1120, discloses the development and optimization of a composition of a self-microemulsifying drug delivery system (SMEDDS) of albendazole.

There continues to be a need for cadotril products having the attributes discussed above.

### SUMMARY OF THE INVENTION

The present invention relates to methods of treatment using cadotril compositions. In one embodiment, the method involves the use of a composition comprising racecadotril, at least one surfactant and a lipid.

In accordance with the present invention, cadotril compositions exhibit improved absorption, rapid onset of action and increased bioavailability.

In one embodiment, the amount of racecadotril is from about 10 mg to about 200 mg per dose. Preferably, the amount of racecadotril is about 3 mg, about 10 mg, about 30 mg, about 50 mg, about 100 or about 150 mg mg/dose.

In accordance with an embodiment, racecadotril is administered 1×/day, 2×/day, 3×/day or 4×/day.

Other features and advantages of the present invention will be apparent from the detailed description of the invention and from the claims.

### BRIEF DESCRIPTION OF THE FIGURES

FIG. 1 shows pK profiles for Tiorfast® (150 mg) and Formulas 1A-5A.

FIGS. 2-7 show individual pK profiles for Tiorfast® (150 mg) and Formulas 1A-5A, respectively.

FIG. 8 is a graph showing droplet size vs. emulsifier (lipid) for Formulas 1A-5A.

FIG. 9 shows surface plot of AUC ratio vs. droplet size vs. emulsifier (lipid).

### DETAILED DESCRIPTION OF THE INVENTION

It is believed that one skilled in the art can, based upon the description herein, utilize the present invention to its fullest extent. The following specific embodiments are to be construed as merely illustrative, and not as limiting the remainder of the disclosure in any way whatsoever.

Unless defined otherwise, all technical and scientific terms used herein have the same meaning as commonly understood by one of ordinary skill in the art to which the invention belongs. As used herein, all percentages are by weight unless otherwise specified. In addition, all ranges set forth herein are meant to include any combinations of values between the two endpoints, inclusively.

### Definitions

“AUC” as used herein means, for any given drug, the “area under the concentration-time curve” from dosing or activation of the drug to a time point, often calculated by the trapezoidal rule. AUC is a parameter showing the cumulative plasma concentration of a drug over time, and is an indicator of the total amount and availability of a drug in the plasma.

$${AUC}_{0}^{∞} = {AUC}_{0}^{t} + {AUC}_{t}^{∞} = {AUC}_{0}^{t} + \frac{c ( t )}{{k}_{e l}} ;$$

$$where$$

$${k}_{el} = \frac{ln 2}{{t}_{1 / 2}}$$

“Cmax” as used herein means the maximum (or peak) concentration that a drug achieves in tested area after the drug has been administrated and prior to the administration of a second dose.

“MRT” or mean residence time is the average amount of time that a drug spends in the body.

$$MRT = \frac{AUMC}{AUC}$$

$$where$$

$$AUMC = {\int }_{0}^{∞} t * c ( t ) d t$$

$$and$$

$$AUC = {\int }_{0}^{∞} c ( t ) d t$$

As used herein, “pharmacodynamics” or “PD” is the study of the relationship between drug concentration at the site of action and the resulting effect.

As used herein, “pharmacokinetics” or “PK” is the study of the time course of drug absorption, distribution, metabolism and excretion.

As used herein, a drug “release rate” refers to the quantity of drug released from a dosage form per unit time, e.g., milligrams of drug released per hour (mg/hr). Drug release rates are calculated under in vitro dosage form dissolution testing conditions known in the art. As used herein, a drug release rate obtained at a specified time “following administration” refers to the in vitro drug release rate obtained at the specified time following commencement of an appropriate dissolution test, e.g., those set forth in USP 24 (United States Pharmacopeia 24, United States Pharmacopeia Convention, Inc., Rockville, Md.).

“Semi-solid dosage forms” shall mean dosage forms which are highly viscous and share some of the properties of liquids, including but not limited to (1) having the ability to substantially conform to something that applies pressure to it and causes its shape to deform; and (2) lacking the ability to flow as easily as a liquid. Semi-solid dosage forms also share some of the properties of solids, including but not limited to having a higher density and a defined shape. Semi-solids may nonexclusively include gels, chewy dosage forms, pectin based chewy forms, confectionery chewy forms, moldable gelatin type of forms.

“Solid dosage forms” shall mean dosage forms which are substantially solid at room temperature and have a density of at least about 0.5 g/cc. Solid dosage forms may non exclusively include, agglomerated tablets, capsule-like medicaments, powder or granule filled capsules, powder or granule filled sachets, compressed tablets, coated tablets, chewable dosage forms, and fast-dissolving dosage forms.

“T_{1/2}” shall mean the amount of time required for one half of the total amount of a drug in a biological system to be degraded by biological processes.

“Tₘₐₓ” shall mean the amount of time after administration of a drug when the maximum plasma concentration is reached.

“Elimination rate constant” (abbreviated as “kₑₗ” and sometimes kₑ) is the first order rate constant describing drug elimination from the body. This is an overall elimination rate constant describing removal of the drug by all elimination processes including excretion and metabolism. Metabolites are different chemical entities and have their own elimination rate constant. The elimination rate constant is the proportionality constant relating the rate of change drug concentration and concentration or the rate of elimination of the drug and the amount of drug remaining to be eliminated.

By “delayed release,” it is meant that, after administration, there is at least one period of time when an active ingredient is not being released from the dosage form, i.e., the release of the active ingredient(s) occurs at a time other than immediately following administration.

As used herein, “dissolution medium” shall mean any suitable liquid environment in which the dosage form of the present invention can be dissolved, such as, for example, the in vitro dissolution media used for testing of the product, or gastro-intestinal fluids. Suitable in vitro dissolution media used for testing the dissolution of the active ingredient or ingredients from the suspension dosage form of the present invention include those described in the United States Pharmacopeia.

A “dosage”, “dosage form” or “dose” as used herein means the amount of a pharmaceutical formulation comprising therapeutically active agent(s) administered at a time. “Dosage”, “dosage form” or “dose” includes administration of one or more units of pharmaceutical formulation administered at the same time.

By “extended release,” it is meant that, after administration, an active ingredient is released from the dosage form in a substantially continuous, regulated manner, and the time for complete release, i.e., depletion, of the active ingredient from the dosage form is longer than that associated with an immediate release dosage form of the same. Types of extended release include controlled, sustained, prolonged, zero-order, first-order, pulsatile, and the like.

As used herein, “immediate release” means that the dissolution characteristics of at least one active ingredient meet USP specifications for immediate release tablets containing that active ingredient. An active ingredient having an immediate release property may be dissolved in the gastrointestinal contents, with no intention of delaying or prolonging the dissolution of the active ingredient.

“Liquid dosage forms” may nonexclusively include dispersions, suspensions, solutions or elixirs, wherein one or more of the active ingredients is dissolved, partially dissolved or in an undissolved or suspended state.

As used herein, a drug “release rate” refers to the quantity of drug released from a dosage form per unit time, e.g., milligrams of drug released per hour (mg/hr). Drug release rates are calculated under in vitro dosage form dissolution testing conditions known in the art. As used herein, a drug release rate obtained at a specified time “following administration” refers to the in vitro drug release rate obtained at the specified time following commencement of an appropriate dissolution test, e.g., those set forth in USP 24 (United States Pharmacopeia 24, United States Pharmacopeia Convention, Inc., Rockville, Md.).

“Therapeutic effect,” as used herein, shall mean any effect or action of an active ingredient intended to diagnose, treat, cure, mitigate, or prevent disease, or affect the structure or any function of the body.

As used herein, a “microemulsion” refers to a liquid mixture of a lipid, water and at least one surfactant. A microemulsion is characterized by its clear, thermodynamically stable, and isotropic appearance.

As used herein, “stable” refers to a composition that is clear to the naked eye and substantially free of chemical degradation of racecadotril, substantial color change, turbidity or oily globules. No phase separation should be observed in either aqueous and/or non-aqueous components for at least about 3 months at 40° C. More preferably, no phase separation should be observed in either aqueous and/or non-aqueous components for at least about 6 months at 40° C. In one embodiment, the total chemical degradant products of racecadotril should be less than 0.5 percent by weight (wt. %), e.g. less than 0.2 wt. % based on the total wt. % of racecadotril when stored at 3 months and 40° C. In another embodiment, the total chemical degradant products of racecadotril should be less than 0.5 percent by weight (wt. %), e.g., less than 0.2 wt. % based on the total wt. % of racecadotril when stored at 6 months and 40° C. The percent degradation products are determined by calculating the % peak area of the degradation product peak areas relative to the peak areas of the Racecadotril peaks in the HPLC chromatograms. In one embodiment, the total chemical degradant products of racecadotril should be less than 0.5% of racecadotril, e.g. less than 0.2% based on of the total % of racecadotril when stored at 3 months and 40° C.

As used herein, “self-microemulsifying drug delivery systems” (SMEDDS) are mixtures of oils, surfactants, and sometimes cosolvents. SMEDDS can be used for formulating systems to improve the oral absorption of highly lipophilic compounds. SMEDDS emulsify spontaneously using gentle agitation to produce fine oil-in-water emulsions when introduced into an aqueous phase. A drug in an SMEDDS appears in a small droplet size and exhibits increased dissolution and permeability. SMEDDS may be formulated for liquid or solid use. For solid use, the solids are packaged in capsules or tablets. Liquid filled or semi-solid filled capsules are a preferred dosage form by certain consumers, due to the perception of speed, visual appearance of the drug composition and ease of swallowing.

Various studies have shown racecadotril to be efficacious in reducing the symptoms of diarrhea. One benefit of using racecadotril over other remedies is that racecadotril has been shown to have fewer side effects such as post-treatment constipation.

Racecadotril has low water solubility, of about 10 micrograms/ml at room temperature conditions.

Racecadotril is included in the microemulsion composition in an amount from about 0.01 wt. % to about 24.0 wt. % per 100 ml of the emulsion composition. Preferably, the racecadotril is about 1.0 wt. % to about 18.0 wt. %, and more preferably, about 2.0 wt. % to about 12.0 wt. % per 100 ml of the emulsion composition, and even more preferably, about 3.0 wt. % to about 10.0 wt. % per 100 ml of the emulsion composition. In one embodiment, the racecadotril is about 4.0 wt. % to about 24.0 wt. % per 100 ml of the emulsion composition. In another embodiment, the racecadotril is about 4.0 wt. % to about 18.0 wt. % per 100 ml of the emulsion composition. In yet another embodiment, the racecadotril is about 4.0 wt. % to about 12.0 wt. % per 100 ml of the emulsion composition. In still yet another embodiment, the racecadotril is about 4.0 wt. % to about 10.0 wt. % per 100 ml of the emulsion composition.

The microemulsion composition includes at least one surfactant. The surfactant may be, for example, a nonionic surfactant, cationic surfactant, anionic surfactant, or mixtures thereof.

Suitable surfactants include, for example, water-insoluble surfactants having a hydrophilic-lipophilic balance (HLB) value less than 12 and water-soluble surfactants having a HLB value greater than 12. Surfactants that have a high HLB and hydrophilicity, aid the formation of oil-water droplets. The surfactants are amphiphilic in nature and are capable of dissolving or solubilizing relatively high amounts of hydrophobic drug compounds.

Non-limiting examples, include, Tween, Dimethylacetamide (DMA), Dimethyl sulfoxide (DMSO), Ethanol, Glycerin, N-methyl-2-pyrrolidone (NMP), PEG 300, PEG 400, Poloxamer 407, Propylene glycol, Phospholipids, Hydrogenated soy phosphatidylcholine (HSPC), Distearoylphosphatidylglycerol (DSPG), L-α-dimyristoylphosphatidylcholine (DMPC), L-α-dimyristoylphosphatidylglycerol (DMPG), Polyoxyl 35 castor oil (CREMOPHOR EL, CREMOPHOR ELP), Polyoxyl 40 hydrogenated castor oil (Cremophor RH 40), Polyoxyl 60 hydrogenated castor oil (CREMOPHOR RH 60), Polysorbate 20 (TWEEN 20), Polysorbate 80 (TWEEN 80), d-α-tocopheryl polyethylene glycol 1000 succinate (TPGS), Solutol HS-15, Sorbitan monooleate (SPAN 20), PEG 300 caprylic/capric glycerides (SOFTIGEN 767), PEG 400 caprylic/capric glycerides (LABRASOL), PEG 300 oleic glycerides (LABRAFIL M-1944CS), Polyoxyl 35 Castor oil (ETOCAS 35), Glyceryl Caprylate (Mono- and Diglycerides) (IMWITOR), PEG 300 linoleic glycerides (LABRAFIL M-2125CS), Polyoxyl 8 stearate (PEG 400 monosterate), Polyoxyl 40 stearate (PEG 1750 monosterate), Peppermint oil, and combinations thereof.

Additionally, suitable surfactants include, for example, polyoxyethylene derivative of sorbitan monolaurate such as polysorbate, caprylcaproyl macrogol glycerides, polyglycolyzed glycerides, and the like.

In one embodiment, the surfactant is a combination of polyoxyl 35 castor oil and glyceryl caprylate (mono- and diglycerides) NF.

In the composition, the total weight percent of surfactant(s) is from about 1 wt. % to about 95 wt. % per 100 ml of the microemulsion composition. Preferably, the surfactant is about 25 wt. % to about 95 wt. %, and more preferably, about 30 wt. % to about 90 wt. % per 100 ml of the microemulsion composition. In one embodiment, the surfactant is about 45 wt. % to about 90 wt. % per 100 ml of the microemulsion composition.

A lipid is another essential component of the composition. The lipid aids in solubilizing the racecadotril and also facilitates the self-emulsification process. Suitable lipids include, for example, vegetable oils (modified and/or hydrolyzed), long-chain triglycerides and medium-chain triglycerides (MCTs) having different degrees of saturation, and combinations thereof may be used.

In addition, monoglyceride, diglyceride, and/or triglyceride emulsifiers (fats and oils) that are lipophilic and insoluble in water (available from Abitec Corporation, sold under the tradename CAPMUL®) may be used as the lipid. For example, Beeswax, Oleic acid, Soy fatty acids, d-α-tocopherol (Vitamin E), Corn oil mono-di-tridiglycerides, Medium chain (C8/C10) mono- and diglycerides, Long-chain triglycerides, Castor oil, Corn oil, Cottonseed oil, Olive oil, Peanut oil, Peppermint oil, Safflower oil, Sesame oil, Soybean oil, Hydrogenated soybean oil, Hydrogenated vegetable oils, Medium-chain triglycerides, Caprylic/capric triglycerides derived from coconut oil, palm seed oil, and combinations thereof.

The lipid is included in the composition in an amount from about 0.01 wt. % to about 60 wt. % per 100 ml of the emulsion composition. Preferably, the lipid is about 0.1 wt. % to about 50 wt. %. In another embodiment, the lipid is about 1 wt. % to about 20 wt. % per 100 ml of the emulsion composition, more preferably, about 1 wt. % to about 15 wt. % per 100 ml of the emulsion composition, and even more preferably, about 1 wt. % to about 10 wt. % per 100 ml of the emulsion composition. In one particular embodiment, the lipid is from about 1 wt. % to about 2 wt. % per 100 ml of the emulsion composition.

It is desirable to minimize the amount of water in the composition. The amount of water in the composition will be largely determined by the water content of each component that is included in the composition. In one embodiment, the water content of the composition is less than about 3.5 wt. % based on the total wt. % of the composition. In another embodiment, the water content of the composition is less than about 2.5 wt. % based on the total wt. % of the composition. In yet another embodiment, the water content of the composition is less than about 0.5 wt. % based on the total wt. % of the composition. In still yet another embodiment, the water content of the composition is less than about 0.2 wt. % based on the total wt. % of the composition.

Optionally, a variety of ingredients may be included in the emulsion composition.

Any coloring agent suitable for use in a food or pharmaceutical product may be used. Typical coloring agents include, for example, azo dyes, quinopthalone dyes, triphenylmethane dyes, xanthene dyes, indigoid dyes, iron oxides, iron hydroxides, titanium dioxide, natural dyes, and mixtures thereof. More specifically, suitable colorants include, but are not limited to patent blue V, acid brilliant green BS, red 2G, azorubine, ponceau 4R, amaranth, D&C red 33, D&C red 22, D&C red 26, D&C red 28, D&C yellow 10, FD&C yellow 5, FD&C yellow 6, FD&C red 3, FD&C red 40, FD&C blue 1, FD&C blue 2, FD&C green 3, brilliant black BN, carbon black, iron oxide black, iron oxide red, iron oxide yellow, titanium dioxide, riboflavin, carotenes, antyhocyanines, turmeric, cochineal extract, clorophyllin, canthaxanthin, caramel, betanin, and mixtures thereof.

Similarly, a flavor may be included in the emulsion composition. The amount of flavor added to the composition is dependent upon the desired taste characteristics.

The composition may contain other ingredients or components, such as aromas; sweeteners such as sucralose, sorbitol, high fructose corn syrup, sugar, and the like; viscosity modifiers such as xanthan gum; preservatives such as sodium benzoate NF, buffers such as citric acid and/or sodium chloride; or mixtures thereof.

The emulsion composition may be made by any method known to those skilled in the art so long as it results in the desired composition.

Suitable methods include, for example, combining each ingredient in a mixing kettle, where the ingredients may be added sequentially or in any manner so long as the intended result is achieved. Moreover, the mixing action should be sufficient to incorporate each ingredient into the composition.

The stability of the lipid-based formulation is based on degradation analysis of racecadotril when stored at 40° C. and analyzed at various time points.

The self-emulsifying emulsion can be characterized by quantifying the droplet size, viscosity, turbidity, and polydispersity index.

The lipid-based formulation was prepared as a self-emulsifying emulsion with 0.1N HCl in order to determine droplet size by dynamic light scattering (DLS) and evaluating oversaturation by observing precipitation over time.

In one embodiment, the microemulsion composition is administered as a packaged emulsion for direct oral consumption. In another embodiment, the microemulsion composition is administered in an oral soft gelatin capsule containing the microemulsion composition. In yet another embodiment, the microemulsion composition is administered in a multiple of microgel beads containing the microemulsion composition. In still yet another embodiment, the microemulsion composition is administered in a hard gelatin capsule containing the microemulsion composition. When the microemulsion composition is contained in the hard gelatin capsule, the hard gelatin capsule may be banded. In still yet another embodiment, the microemulstion composition is administered in a suppository or enema containing the microemulsion composition.

In one embodiment the microemulsion composition of the present invention is adsorbed onto an inert adsorbant. In this embodiment the adsorbant and microemulsion are incorporated into a solid dosage form such as a compressed tablet, hard shell capsule, sachet, powder, granule or caplet.

The inert adsorbent is, for example, laponite, bentonite, clays, veegum (magnesium aluminosilicate), Neusilin®, Fuji Chemical Industries (magnesium aluminometasilicate), Florite®, Tomita Pharmaceutical (porous calcium silicate), and dicalcium phosphate and tricalcium phosphate, Syloid®, Grace Materials Technologies (mesoporous silicon dioxide), and mixtures thereof. Optionally, the microemulsion composition may comprise a second active ingredient. In one embodiment the second active ingredient is a digestive health active ingredient. Non-limiting examples, include, for example, laxatives, antacids, proton pump inhibitors, anti-gas agents, antiemetics, H2 blockers, or a second antidiarrheal agent.

In one embodiment, the second active ingredient is incorporated into the microemulsion composition. In another embodiment, the second active ingredient is present in another portion of the dosage form composition which is separate from the microemulsion composition. In yet another embodiment, the second active ingredient is microencapsulated.

Suitable anti-gas agents include, but are not limited to simethicone.

Suitable additional antidiarrheal agents include, but are not limited to loperamide.

In one embodiment, the microemulsion composition includes about 8.0 wt. % to about 10.0 wt. % racecadotril, about 88 wt. % to about 91 wt. % of surfactant in total, about 1 wt. % to about 2 wt. % lipid, wherein each wt. % is based upon 100 ml of the composition.

In another embodiment, the microemulsion composition includes about 0.01 wt. % to about 24.0 wt. % racecadotril, about 1 wt. % to about 95 wt. % of surfactant in total, about 0.01 wt. % to about 60 wt. % lipid, wherein each wt. % is based upon 100 ml of the composition.

In yet another embodiment, the microemulsion composition includes about 3.0 wt. % to about 7.0 wt. % racecadotril, about 40 wt. % to about 53 wt. % of surfactant in total, about 40 wt. % to about 53 wt. % lipid, wherein each wt. % is based upon 100 ml of the composition.

The microemulsion composition may be delivered in any suitable delivery system. For example, in one embodiment, the microemulsion composition is delivered orally. In another embodiment, the microemulsion composition is delivered in a soft shell dosage form. In still another embodiment, the microemulsion composition is delivered in a hard shell dosage form. In still yet another embodiment, a tablet dosage form is used to deliver the microemulsion composition.

In addition, the droplet size of the composition was measured using a Horiba SZ-100 Nanoparticle Size Analyzer by dynamic light scattering (DLS) at a scattering angle of 90 degrees. Samples were kept in a temperature control chamber at 25° C. during measurement Immediately prior to measurement the instrument performance was checked with a nominal 100 nm polystyrene latex (PSL) size standard in 10 mM NaCl. Count rates for these measurements ranged from 1 million to 3 million counts per second. The measurements were performed for one minute each. Data were analyzed using the cumulant technique.

The droplet size was also measured on a Nicomp 380 Nanoparticle Size Analyzer by dynamic light scattering (DLS) with a scattering angle of 90 degrees at Particle Sizing Systems (PSS). All measurements were performed at 23° C. After warming up, the instrument was challenged with a NIST traceable standard (i.e., polystyrene latex) to check for accuracy. A scattering intensity of 150-500 kHz was targeted during sample measurement which lasted for 15 minutes. Data were analyzed using the cumulant technique.

The present invention also includes a method for treating a subject experiencing diarrhea comprising the step of orally administering to the subject a composition comprising racecadotril, at least one surfactant, and a lipid.

The present invention relates to methods of treatment using cadotril compositions, such as racecadotril, dexecadotril and ecadotril compositions.

Racecadotril, dexecadotril and ecadotril are enkephalinase inhibitors with unique intestinal antisecretory activity. The compounds are insoluble in water. Solubility of racecadotril in various media is shown below.

Their bitter taste and degradation profile have rendered formulation challenging. For example, a difficulty in preparing stable suspensions of racecadotril is the risk of hydrolysis of this compound which bears an ester group and can be easily hydrolyzed into easily oxidizable and less active compounds.

Stability of racecadotril in various systems is shown below.

Various studies have shown racecadotril to be efficacious in reducing the symptoms of diarrhea. One benefit of using racecadotril over other remedies is that racecadotril has been shown to have fewer side effects such as post-treatment constipation.

According to another preferred aspect, said treatment comprises oral administration, preferably one to four times a day.

The following examples are provided to further illustrate the compositions and methods of the present invention. It should be understood that the present invention is not limited to the examples described.

### EXAMPLES

### Example 1

### Concentrated Racecadotril Lipid Composition: for use in Liquid Filled Gelatin Capsule

TABLE 1
Racecadotril Lipid Based Composition as a percentage of the composition:
Triglyceride Type 1

| | Formula 1 | Formula 3 | Formula 5 |
|---|---|---|---|
| Ingredient | (% w/w) | (% w/w) | (% w/w) |
| Racecadotril | 9.60 | 9.31 | 8.34 |
| Polyoxyl 35 Castor oil¹ | 79.55 | 52.60 | 27.50 |
| Glyceryl Caprylate | 9.04 | 36.27 | 62.33 |
| (Mono- and Diglycerides) NF² | | | |
| Medium Chain Triglycerides³ | 1.81 | 1.81 | 1.83 |
| Total | 100 | 100 | 100 |
| Racecadotril Assay (mg/mL) | 96.04 | 93.14 | 83.37 |

¹Commercially available from CRODA Healthcare as ETOCAS ® 35 USP/NF, EP, JP
²Commercially available from CREMER as IMWITOR ® 988 USP/NF, EP, JP
³Commercially available from CREMER as MIGLYOL ® 810N (Caprylic/Capric Triglycerides; 70:30/C8:C10) USP/NF, EP, JP

TABLE 2
Racecadotril Lipid Based Composition as a percentage of the composition:
Triglyceride Type 2

| | Formula 2 | Formula 4 | Formula 6 |
|---|---|---|---|
| Ingredient | (% w/w) | (% w/w) | (% w/w) |
| Racecadotril | 9.47 | 8.98 | 8.33 |
| Polyoxyl 35 Castor oil¹ | 79.67 | 52.79 | 27.50 |
| Glyceryl Caprylate | 9.05 | 36.41 | 62.33 |
| (Mono- and Diglycerides) NF² | | | |
| Medium Chain Triglycerides³ | 1.81 | 1.82 | 1.83 |
| Total | 100 | 100 | 100 |
| Racecadotril Assay (mg/mL) | 94.68 | 89.77 | 83.34 |

¹Commercially available from CRODA Healthcare as ETOCAS ® 35 USP/NF, EP, JP
²Commercially available from CREMER as IMWITOR ® 988 USP/NF, EP, JP
³Commercially available from CREMER as MIGLYOL ® 812N (Caprylic/Capric Triglycerides; 60:40/C8:C10) USP/NF, EP, JP

Utilizing the materials in Table 1 and Table 2, the following mixing steps were taken to form the microemulsion. A total of 6 mixtures were prepared including 3 ratios, with each prepared with MIGLYOL 810N (Table 1) and MIGLYOL 812N (Table 2).

Step 1: In a suitable vessel, a mixture of the Polyoxyl 35 Castor oil (ETOCAS® 35), Glyceryl Caprylate (IMWITOR® 988) and Medium Chain triglycerides (MIGLYOL® 810N & 812N) was prepared in three separate mixtures in the following weight ratios: 88:10:2 (Ratio 1), 58:40:2 (Ratio 2), and 30:68:2 (Ratio 3).

Step 2: The mixture(s) from Step 1 were mixed utilizing a vortex mixer.

Step 3: The Racecadotril was slowly added to the mixture(s) from Step 2 utilizing the vortex mixer, and mixed for 5 minutes.

Step 4: The mixture from Step 3 was placed into a laboratory shaker and mixed for 36 hours until a clear solution was formed.

### Stability of Racecadotril Lipid Formulation

The chemical stability of the formulations prepared in Example 1 was examined for racecadotril degradation when stored for 40.1 weeks at 40° C. in sealed glass bottles, and is shown in Table 3.

TABLE 3
Stability Data for lipid-based Formulations:
Formula 1, Formula 3, Formula 5

| | RAC (%) | | | Benzyl Alcohol (%) | | | Impurity C (%) | | | Impurity G (%) | | |
|---|---|---|---|---|---|---|---|---|---|---|---|---|
| Time | Form. 1 | Form. 3 | Form. 5 | Form. 1 | Form. 3 | Form. 5 | Form. 1 | Form. 3 | Form. 5 | Form. 1 | Form. 3 | Form. 5 |
| Initial | 99.95 | 99.94 | 99.95 | ND | ND | ND | ND | ND | ND | ND | ND | ND |
| 6 wk | 99.67 | 99.23 | 99.00 | 0.06 | 0.29 | 0.29 | ND | ND | 0.01 | 0.01 | 0.02 | 0.02 |
| 12 wk | 99.46 | 98.45 | 98.61 | 0.13 | 0.48 | 0.32 | ND | ND | 0.04 | 0.01 | 0.02 | 0.02 |
| 16 wk | 99.15 | 97.82 | 97.79 | 0.18 | 0.66 | 0.49 | ND | ND | 0.10 | 0.02 | 0.02 | 0.02 |
| 40.1 wk | 98.86 | 96.89 | 96.86 | 0.26 | 0.85 | 0.74 | 0.07 | 0.11 | 0.26 | 0.02 | 0.09 | 0.01 |

Formula 2, Formula 4, Formula 6

| | RAC (%) | | | Benzyl Alcohol (%) | | | Impurity C (%) | | | Impurity G (%) | | |
|---|---|---|---|---|---|---|---|---|---|---|---|---|
| Time | Form. 2 | Form. 4 | Form. 6 | Form. 2 | Form. 4 | Form. 6 | Form. 2 | Form. 4 | Form. 6 | Form. 2 | Form. 4 | Form. 6 |
| Initial | 99.95 | 99.94 | 99.94 | ND | ND | ND | ND | ND | ND | ND | ND | ND |
| 6 wk | 99.66 | 99.11 | 98.94 | 0.05 | 0.30 | 0.34 | ND | ND | ND | 0.02 | 0.02 | 0.02 |
| 12 wk | 99.41 | 98.37 | 98.49 | 0.12 | 0.52 | 0.45 | ND | ND | ND | 0.02 | 0.02 | 0.02 |
| 16 wk | 99.09 | 97.78 | 97.86 | 0.15 | 0.65 | 0.57 | ND | ND | 0.05 | 0.02 | 0.02 | 0.02 |
| 40.1 wk | 98.74 | 96.95 | 96.85 | 0.27 | 0.87 | 0.79 | 0.14 | 0.08 | 0.22 | 0.02 | 0.06 | ND |

There was no Impurity A, thiorphan, or Impurity E in Form. 1, Form. 2, Form. 3, Form. 4, Form. 5, Form. 6
ND: not detectable

Formula:

1. 88% Super Refined Etocas 35, 10% Imwitor 988, 2% Miglyol 810N (Ratio 1)

2. 88% Super Refined Etocas 35, 10% Imwitor 988, 2% Miglyol 812N (Ratio 1)

3. 58% Super Refined Etocas 35, 40% Imwitor 988, 2% Miglyol 810N (Ratio 2)

4. 58% Super Refined Etocas 35, 40% Imwitor 988, 2% Miglyol 812N (Ratio 2)

5. 30% Super Refined Etocas 35, 68% Imwitor 988, 2% Miglyol 810N (Ratio 3)

6. 30% Super Refined Etocas 35, 68% Imwitor 988, 2% Miglyol 812N (Ratio 3)

ND—Not detectable

### Ingredient:

A. Super Refined Etocas 35 (NF, EP, JP):
   Manufactured by CRODA Health Care
   Polyoxyl 35 Castor Oil
   HLB value of ˜14
B. Imwitor 988: Medium Chain Partial Glycerides
   Manufactured by CREMER
   Glyceryl Caprylate (Mono- and Diglycerides)
   Melting Point ˜25° C.
   HLB value of ˜4
C. Imwitor 742: Medium Chain Partial Glycerides
   Manufactured by CREMER
   Caprylic/Capric Glycerides
   Melting Point ˜25° C.
   HLB value of ˜3-4
D. Miglyol: Medium Chain Triglycerides (MCT Oils, Fractionated Coconut Oil)
   Manufactured by CREMER
   Caprylic (C8)/Capric (C10) Triglycerides
   810N-70:30 C8/C10 blend
   812N-60:40 C8/C10 blend
      Conversion based on the density of each formula:
Formula 1/Formula 2: 1.042 g/ml
Formula 3/Formula 4: 1.028 g/ml
Formula 5/Formula 6: 1.016 g/ml
   Water Content (% w/w):

| | |
|---|---|
| Racecadotril | 0.5% |
| Super Refined Etocas 0-3% (EP): | 0% |
| Super Refined Etocas 0-1% (JP): | 0% |
| Imwitor 988: | 0.2% |
| Miglyol 810N: | 0.01% |
| Miglyol 812N: | 0.01% |

| | Water Content |
|---|---|
| Formula | (% w/w) |
| 1 | 0.02 |
| 2 | 0.02 |
| 3 | 0.08 |
| 4 | 0.08 |
| 5 | 0.13 |
| 6 | 0.13 |
| 7 | 0.09 |
| 8 | 0.09 |
| 9 | 0.10 |
| 10 | 0.10 |

### Example 2

### Concentrated Racecadotril Lipid Composition: for use in Liquid Filled Gelatin Capsule

TABLE 4

| | Formula 7 | Formula 8 |
|---|---|---|
| Ingredient | (% w/w)^{a} | (% w/w) |
| Racecadotril | 4.61 | 4.25 |
| Glyceryl Caprylate (Mono- and Diglycerides) NF¹ | 47.95 | 48.00 |
| Medium Chain Triglycerides² | 47.44 | — |
| Medium Chain Triglycerides³ | — | 47.75 |
| Total | 100 | 100 |
| Racecadotril Assay (mg/mL) | 46.11 | 42.49 |

¹Commercially available from CREMER as IMWITOR 742 ® USP/NF, EP, JP
²Commercially available from CREMER as MIGLYOL ® 810N (Caprylic/Capric Triglycerides; 70:30/C8:C10) USP/NF, EP, JP
³Commercially available from CREMER as MIGLYOL ® 812N (Caprylic/Capric Triglycerides; 60:40/C8:C10) USP/NF, EP, JP

TABLE 5

| | Formula 9 | Formula 10 |
|---|---|---|
| Ingredient | (51.5:48.5)^{a} | (51.4:48.6) |
| Racecadotril | 5.28 | 5.59 |
| Glyceryl Caprylate (Mono - and | 48.83 | 48.54 |
| Diglycerides) NF¹ | | |
| Medium Chain Triglycerides² | 45.90 | — |
| Medium Chain Triglycerides³ | — | 45.87 |
| Total | 100 | 100 |
| Racecadotril Assay (mg/mL) | 52.78 | 55.93 |

¹Commercially available from CREMER as IMWITOR 988 ® USP/NF, EP, JP
²Commercially available from CREMER as MIGLYOL ® 810N (Caprylic/Capric Triglycerides; 70:30/C8:C10) USP/NF, EP, JP
³Commercially available from CREMER as MIGLYOL ® 812N (Caprylic/Capric Triglycerides; 60:40/C8:C10) USP/NF, EP, JP

Testing Methods:

### Sample Preparation: (in Acetonitrile)

1. Pipet 1 mL of Racecadotril lipid solution into a 100 mL volumetric flask (V.F.)
2. Dilute to volume with Acetonitrile. Add about 20 mL of Dimethylacetamide if necessary.
3. Further dilute the sample solution to about 0.1 mg/mL with acetonitrile if necessary.

### Sample Analysis

Inject reference standards (0.1 mg/mL of Racecadotril in Acetonitrile) and samples onto a suitable HPLC system under conditions similar to those suggested below. Parameters may be modified to optimize chromatography.
Determine the assay of Racecadotril using the Racecadotril peak areas of the sample solutions under test in comparison with the Racecadotril peak areas of the standard solution. The degradation products levels are determined by % peak area relative to the Racecadotril peak.

### Chromatographic Conditions (European Pharmacopoeia Racecadotril Method):

| | |
|---|---|
| Column: | Phenomenex Luna 5 μm C18 (2), 100 Å; |
| | 250 mm × 4.6 mm ID |
| | (Column ID in EP is 4.0 mm) |
| Column heater: | 30° C. |
| Wavelength: | 210 nm |
| Inj. Vol.: | 10 μL |
| Flow rate: | 1 mL/min |

### Gradient Table:

| Time (min) | flow | % A | % B |
|---|---|---|---|
| Initial | 1.0 | 60 | 40 |
| 5 | 1.0 | 60 | 40 |
| 25 | 1.0 | 20 | 80 |
| 35 | 1.0 | 20 | 80 |
| 36 | 1.0 | 60 | 40 |
| 45 | 1.0 | 60 | 40 |

Mobil Phase A: Phosphate buffer, pH 2.5 (Buffer prep: dissolve 1 g of potassium dihydrogen phosphate in water, adjust to pH 2.5 with phosphoric acid, dilute to 1000 mL with water)

### Mobil Phase B: 100% Acetonitrile

### Example 3

### Racecadotril Lipid Composition: Droplet Size

### Procedure

The droplet size was measured on a Horiba SZ-100 Nanoparticle Size Analyzer by dynamic light scattering (DLS) at a scattering angle of 90 degrees. During measurement, samples were kept in a temperature control chamber at 25° C. Immediately prior to measurement the instrument performance was checked with a nominal 100 nm polystyrene latex (PSL) size standard in 10 mM NaCl. Count rates for these measurements ranged from 1 million to 3 million counts per second. The measurements were performed for one minute each. Data were analyzed using the cumulant technique.

### Solubility and Droplet Size for Lipid-Based Formulations

** Formula 1, Formula 3, Formula 5

| | Form. 1 | Form. 3 | Form. 5 |
|---|---|---|---|
| Solubility (mg/mL) | 96.0 | 93.1 | 83.4 |
| Solubility (mg/g) | 92.2 ² | 90.6 ³ | 82.1 ⁴ |
| Droplet Size (nm) ^{*} | 18.1 ⁵ | 25.1 ⁶ | 48.3 ⁷ |
| (Z-avg Diameter) ¹ | | | |

** Formula 2, Formula 4, Formula 6

| | Form. 2 | Form. 4 | Form. 6 |
|---|---|---|---|
| Solubility (mg/mL) | 94.7 | 89.8 | 83.3 |
| Solubility (mg/g) | 90.7 ² | 87.7 ³ | 82.1 ⁴ |
| Droplet Size (nm) ^{*} | 19.4 ⁸ | 25.3 ⁹ | 47.2 ¹⁰ |
| (Z-avg Diameter) ¹ | | | |

^{*} Determined by dynamic light scattering (DLS) with a Horiba SZ-100 Nanoparticle Size Analyzer, average of three determinations (n = 3)
¹ General procedure: 0.08 g of each formulation and 15 mL of 0.1 N HCl were combined and mixed by vortex
² Calculated based on density of 1.042 g/mL
³ Calculated based on density of 1.028 g/mL
⁴ Calculated based on density of 1.016 g/mL
⁵ Concentration of racecadotril ~0.53 mg/mL
⁶ Concentration of racecadotril ~0.55 mg/mL
⁷ Concentration of racecadotril ~0.44 mg/mL
⁸ Concentration of racecadotril ~0.60 mg/mL
⁹ Concentration of racecadotril ~0.43 mg/mL
¹⁰ Concentration of racecadotril ~0.46 mg/mL
** See Example 1 for Formula

The droplet size was also measured on a Nicomp 380 Nanoparticle Size Analyzer by dynamic light scattering (DLS) with a scattering angle of 90 degrees at Particle Sizing Systems (PSS). All measurements were performed at 23° C. After warming up, the instrument was challenged with a NIST traceable standard (i.e., polystyrene latex) to check for accuracy. A scattering intensity of 150-500 kHz was targeted during sample measurement which lasted for 15 minutes. Data were analyzed using the cumulant technique.

**Formula 1, Formula 3, Formula 5

| | Form. 1 | Form. 3 | Form. 5 |
|---|---|---|---|
| Solubility (mg/mL) | 96.0 | 93.1 | 83.4 |
| Solubility (mg/g) | 92.2 ² | 90.6 ³ | 82.1 ⁴ |
| Droplet Size (nm)* | 17.2 ⁵ | 22.9 ⁶ | 56.6 ⁷ |
| (Z-avg Diameter) ¹ | | | |
| Determinations (n=) | 2 | 1 | 1 |

**Formula 2, Formula 4, Formula 6

| | Form. 2 | Form. 4 | Form. 6 |
|---|---|---|---|
| Solubility (mg/mL) | 94.7 | 89.8 | 83.3 |
| Solubility (mg/g) | 90.7 ² | 87.7 ³ | 82.1 ⁴ |
| Droplet Size (nm)* | 17.8 ⁸ | 24.7 ⁹ | 47.4 ¹⁰ |
| (Z-avg Diameter) ¹ | | | |
| Determinations (n=) | 1 | 2 | 2 |

*Determined by dynamic light scattering (DLS) with a Nicomp 380 Nanoparticle Size Analyzer.
¹ General procedure: 0.2 mL of formulation and 4.8 mL of 0.1N HCl were combined and mixed for Formula 1 and 3; 0.1 mL of formulation and 4.9 mL of 0.1N HCl were combined and mixed for Formula 2 and 4; 0.1 mL of formulation and 4.9 mL of 0.1N HCl were combined and mixed, then 2.5 mL of dilution was added to 2.5 mL of 0.1N HCl for Formula 5 and 6.
² Calculated based on density of 1.042 g/mL
³ Calculated based on density of 1.028 g/mL
⁴ Calculated based on density of 1.016 g/mL
⁵ Concentration of racecadotril ~3.84 mg/mL
⁶ Concentration of racecadotril ~3.72 mg/mL
⁷ Concentration of racecadotril ~0.83 mg/mL
⁸ Concentration of racecadotril ~1.89 mg/mL
⁹ Concentration of racecadotril ~1.80 mg/mL
¹⁰ Concentration of racecadotril ~0.83 mg/mL
**See Example 1 for Formula

### Example 4

### Canine Crossover PK Study

### Protocol:

A reference formulation (Tiorfast® (150 mg, (source was 100 mg dose Tiorfast® capsule containing racecadotril and lactose as an excipient; 1.5 times the average fill weight was apportioned into each capsule supplied as reference) and five formulations prepared in accordance with the method set forth in Example 1 and having the formulas set forth in Table 6 below were tested as follows:
1. Six (6) male beagle dogs of similar age (1.5-3 yrs) and body weight (9-11 kg) were selected to receive each of the three formulations.
2. Each dog was injected intramuscularly (IM) with pentagastrin solution ˜30 mins prior to dosing to maintain the stomach pH ˜1.2, which is similar to human.
3. Each dog was administered per os (PO) two capsules (equivalent to 150 mg racecadotril) followed by a dosing flush of 100 mL of sterile water.
4. The washout period between dosing each formulation was 4 days.
5. Blood samples were collected at pre-determined time points (0, 5, 15, 30 min, 1, 2, 4, 6, 8, and 24 hrs) and centrifuged at 4° C. with 3000×g for 5 mins.
6. Plasma samples were transferred into appropriate storage vials and treated with the derivatizing reagent 2-bromo-3-methoxyacetophenone (BMP, 0.5 M in acetonitrile) for 10 mins prior to being immediately frozen on dry ice to stabilize the thiorphan.
7. Plasma samples were then analyzed by LC-MS/MS.
8. Pharmacokinetic parameters (i.e., AUC, Cmax, Tmax, T1/2, Kel, MRT) were calculated with WinNonlin® software using a non-compartmental model.

TABLE 6
Composition of Formulae

| | Formula | Formula | Formula | Formula | Formula |
|---|---|---|---|---|---|
| | 1A | 2A | 3A | 4A | 5A |
| Ingredient | (% w/w) | (% w/w) | (% w/w) | (% w/w) | (% w/w) |
| Racecadotril | 9.40 | 8.86 | 8.04 | 7.79 | 7.40 |
| Polyoxyl 35 Castor oil (Super | 79.71 | 52.85 | 27.58 | 18.44 | 9.26 |
| Refined Etocas ® 35; NF, EP, JP)¹ | | | | | |
| Glyceryl Caprylate NF (Mono-, | 9.06 | 36.47 | 62.52 | 71.91 | 81.44 |
| Di-glycerides; Imwitor ® 988; NF, EP, JP)² | | | | | |
| Medium Chain Triglycerides | 1.83 | 1.82 | 1.86 | 1.86 | 1.90 |
| (Miglyol ® 812N; NF, EP, JP)³ | | | | | |
| Total | 100.00 | 100.00 | 100.00 | 100.00 | 100.00 |

¹HLB value of ~14;
²HLB value of ~4;
³HLB value of ~0

### Results:

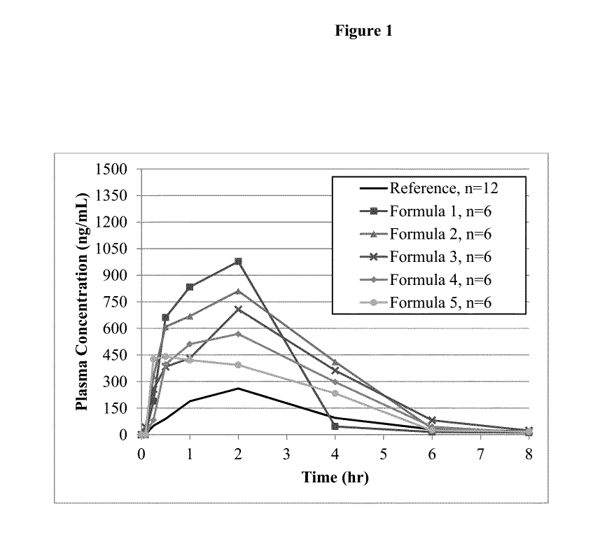

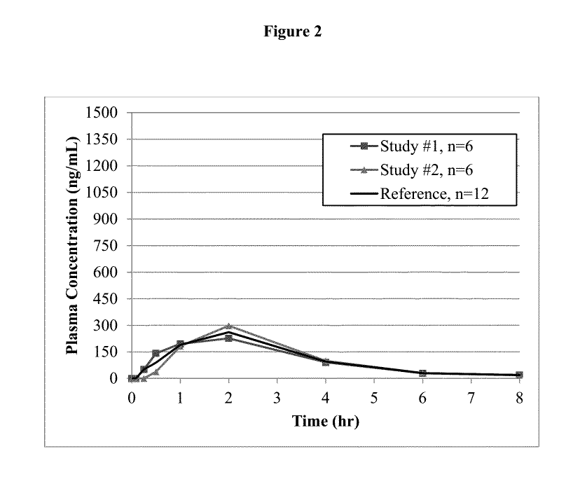

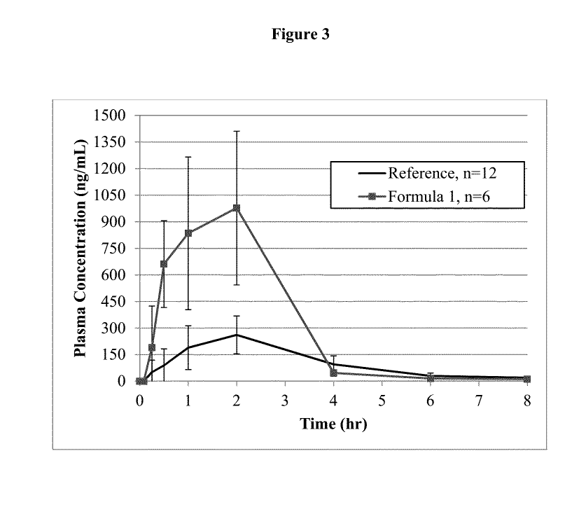

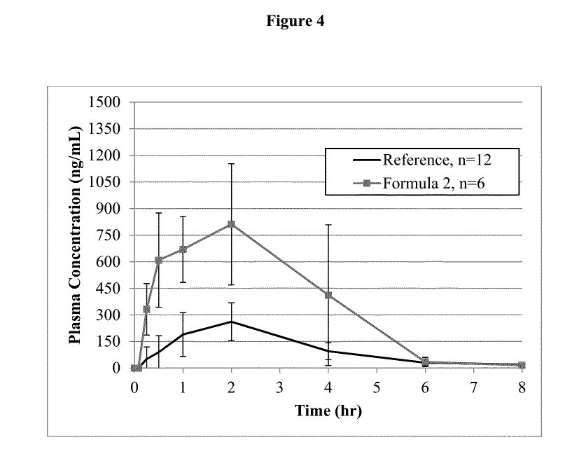

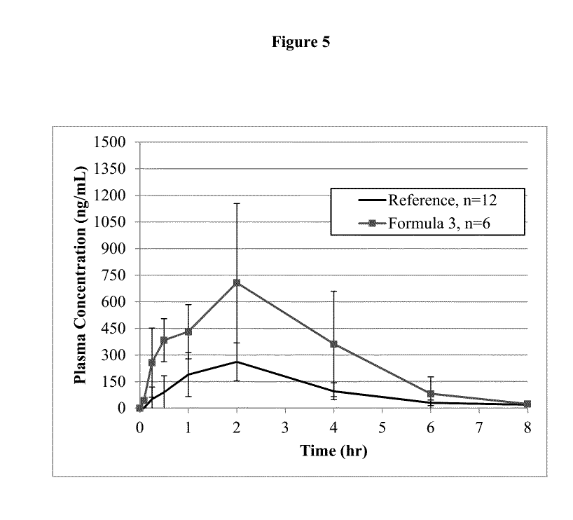

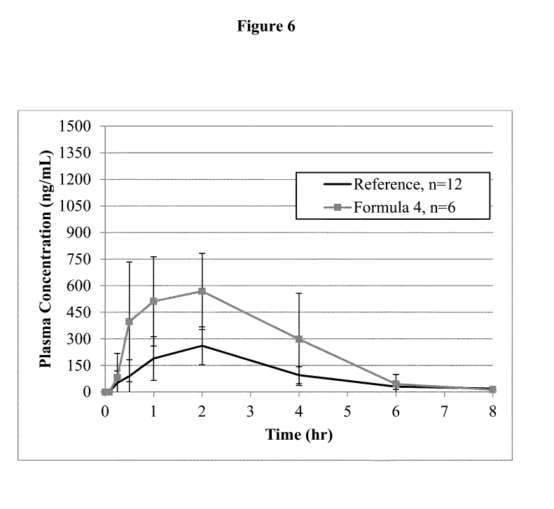

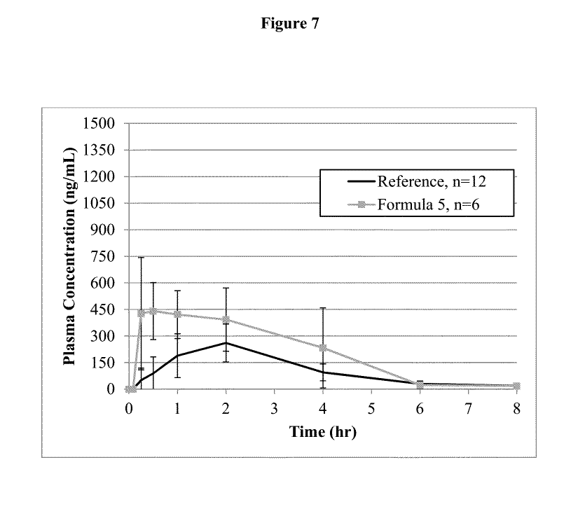

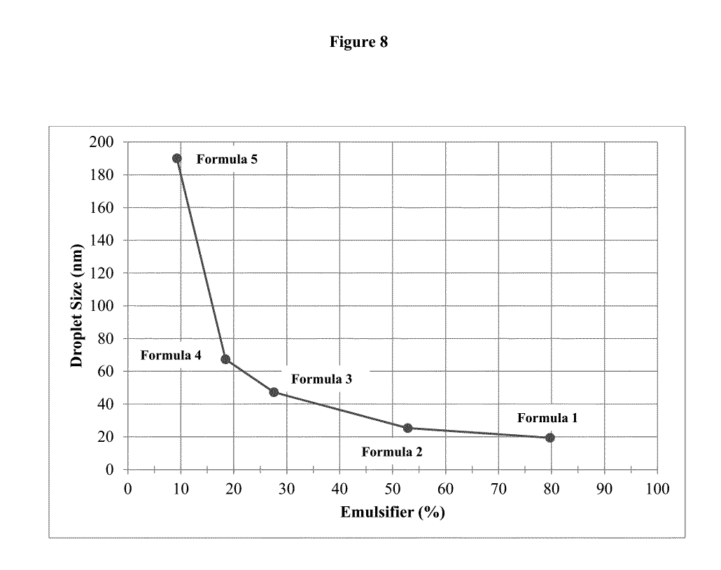

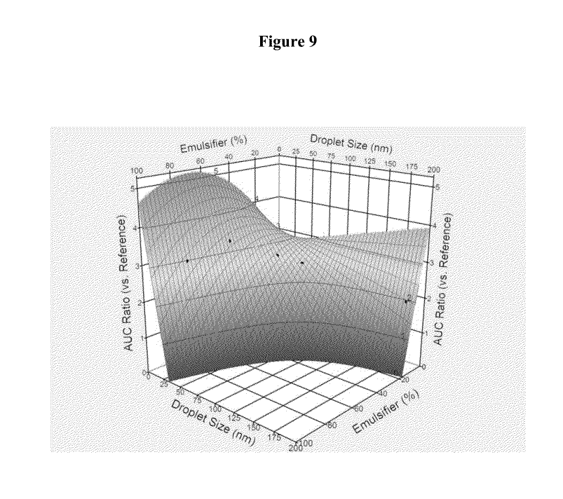

Characteristics of Formulations 1A-5A are set forth below:
Comprised of Super Refined Etocas® 35, Imwitor® 988, and Miglyol® 812N.
Enhanced solubility achieved ranging from 75-100 mg/mL (vs. ˜8 μg/mL).
Three (3) formulations (1A, 2A, 3A) were stable at 40° C. for 10 months with 97-99% potency remaining; stability of two (2) formulations (4A and 5A) was on-going at 40° C. with ˜98% potency remaining at 6 weeks.
All formulae were prepared with 0.1N HCl at various dilution factors to maximize the signal for droplet size determination by DLS with a Horiba SZ-100 Nanoparticle Size Analyzer. Density was determined with a 2-mL specific gravity bottle with a range of (1.0225-1.0663 g/mL. Measurement of nano-emulsion droplet size by dynamic light scatting (DLS) ranged from 19-190 nm; as the amount of surfactant (HLB ∥14) was increased, the resulting droplet size decreased due to the higher degree of emulsification.
All the formulations demonstrated higher AUC (2.0×-3.3×) and Cmax (2.1×-4.4×) as compared to Tiorfast®.
   The results are shown in FIGS. 1-9 and Tables 7 -9 below.

TABLE 7
In-vivo Canine Plasma Concentration (ng/mL) vs. Time (hours)

| Time | | | Formula | | Formula | | Formula | | Formula | | Formula | |
|---|---|---|---|---|---|---|---|---|---|---|---|---|
| (Hr) | Reference^{a} | | 1^{b} | | 2^{b} | | 3^{b} | | 4^{b} | | 5^{b} | |
| 0.083 | ND | (ND) | ND | (ND) | ND | (ND) | 42.3 | (ND) | ND | (ND) | ND | (ND) |
| 0.25 | 50.8 | (67.7) | 189 | (235) | 331 | (145) | 257 | (195) | 82.3 | (136) | 426 | (317) |
| 0.5 | 89.3 | (92.9) | 661 | (245) | 609 | (266) | 383 | (121) | 396 | (338) | 441 | (161) |
| 1 | 189 | (124) | 834 | (431) | 669 | (186) | 431 | (153) | 511 | (252) | 421 | (135) |
| 2 | 261 | (107) | 977 | (434) | 811 | (342) | 707 | (447) | 568 | (215) | 393 | (178) |
| 4 | 95 | (47.9) | 46.2 | (18) | 411 | (397) | 362 | (297) | 297 | (260) | 233 | (226) |
| 6 | 30 | (15.5) | 14.8 | (4.24) | 34.1 | (26) | 81.2 | (95.3) | 44.5 | (54.9) | 23.6 | (19.2) |
| 8 | 19.1 | (7.27) | 11.8 | (3.89) | 14.8 | (7.97) | 23.1 | (11.3) | 12.3 | (4.06) | 17.6 | (7.66) |
| 24 | ND | (ND) | ND | (ND) | ND | (ND) | ND | (ND) | ND | (ND) | ND | (ND) |

Note:
all values reported as ng/mL, Mean (St. Dev.)
^{a}Tiorfast ®, n = 12;
^{b}n = 6

TABLE 8
In-vivo Canine PK Parameters^{e}

| PK | | | Formula | | Formula | | Formula | | Formula | | Formula | |
|---|---|---|---|---|---|---|---|---|---|---|---|---|
| Parameter | Reference^{a} | | 1^{b} | | 2^{b} | | 3^{b} | | 4^{b} | | 5^{b} | |
| Cₘₐₓ (ng/mL) | 286 | (109) | 1249 | (312) | 965 | (211) | 835 | (326) | 644 | (243) | 611 | (118) |
| tₘₐₓ (hr) | 1.9 | (0.8) | 1.4 | (0.7) | 2.2 | (1.0) | 1.9 | (1.3) | 1.9 | (1.2) | 1.6 | (1.4) |
| t_{1/2} (hr) | 2.12 | (0.646) | 1.86 | (0.629) | 0.94 | (0.192) | 1.06 | (0.334) | 0.878 | (0.182) | 0.981 | (0.153) |
| MRT (hr) | 2.71 | (0.505) | 1.72 | (0.193) | 2.32 | (0.595) | 2.55 | (0.628) | 2.37 | (0.696) | 2.11 | (0.717) |
| AUCₜ^{c} | 59.5 | (13.3) | 178 | (37.3) | 195 | (28.8) | 160 | (60.2) | 146 | (51.1) | 117 | (39.1) |
| (hr · kg · ng/mL/mg) | | | | | | | | | | | | |
| AUC_{∞}^{c,d} | 67.6 | (16.3) | 175 | (39.4) | 196 | (29.3) | 154 | (73.2) | 144 | (57.1) | 109 | (37.3) |
| (hr · kg · ng/mL/mg) | | | | | | | | | | | | |

Note:
all values reported as Mean (St. Dev.)
^{a}Tiorfast ®, n = 12;
^{b}n = 6;
^{c}normalized to individual canine weight;
^{d}half-life was not determined for some canines due to a lack of quantifiable data points trailing the Cₘₐₓ or because the terminal elimination phase had an R² of less than 0.85. Therefore, some canines were not included in the AUC_{∞} calculation which caused some AUC_{∞} values to be less than AUCₜ in some cases;
^{e}calculated using WinNonlin software (v. 6.3)

TABLE 9
Property Comparison of Formulae

| | Emulsifier | | Droplet | AUC Ratio |
|---|---|---|---|---|
| | (Etocas | Density | Size, Z-avg | (vs. |
| Formula | 35 ®) | (g/mL)* | (nm)* | Reference)** |
| 1 | 79.71% | 1.0633 (0.0004) | 19.4 (0.06) | 2.992 |
| 2 | 52.85% | 1.0498 (0.0010) | 25.3 (0.27) | 3.277 |
| 3 | 27.58% | 1.0345 (0.0004) | 47.2 (0.47) | 2.689 |
| 4 | 18.44% | 1.0267 (0.0022) | 67.3 (0.93) | 2.454 |
| 5 | 9.26% | 1.0225 (0.0004) | 190.0 (3.26) | 1.966 |

*values reported as Mean (St. Dev.)
**calculated using AUCₜ

The results demonstrate that smaller droplet size resulted in greater exposure and that all formulas had higher AUC (2.0×-3.3×) and Cₘₐₓ (2.1×-4.4×) as compared to marketed Tiorfast® capsules. These positive results indicate that the lipid-based formulae can be leveraged as potential new drug delivery systems to achieve comparable efficacy with a lower dose than Tiorfast® (e.g., (80-100 vs. 300 mg/daily for adults).

### Example 5

### Liquid Self-Emulsifying Racecadotril as a Solid Dosage Form

To formulate a solid dosage form, the liquid formulations were converted to flowable particles and compressed into tablets by two approaches:
Single layer—the flowable particles were blended with various excipients to improve compressibility and formed into tablets.
Compression coating—the flowable particles were compressed into tablets and then coated with an excipient layer in a second compression step.
The liquid formulations were converted to flowable particles by adsorbing the liquid formulations onto highly porous adsorbents with very fine particle size. The porous structure of the adsorbents enables the liquid to be sequestered internally while still remaining flowable. Three (3) adsorbent materials were used:
Syloid® XDP 3150—mesoporous, amorphous silica gel;
Neusilin® US2—amorphous alumnometasilicate; and
Florite® R—calcium silicate.
Adsorption of the liquid formulations onto each material was assessed and used to guide formulation of the single-layer and compression coated tablet.

### Results

Trials with the adsorbent materials were conducted to assess their potential to maximize drug potency.
Three techniques were employed:
Adsorbent loading—Added the liquid formulation to the adsorbents in drop-wise fashion to determine the maximum adsorption level which resulted in flowable particles.
Tablet compression—Determined the maximum adsorption level which allowed tablets to be formed without leaking liquid when the compression force was applied.
Tablet soaking—Tablets of pure adsorbent ( 5/16″, round, flat-faced, bevel edge, 0.5 tons) were soaked in the liquid formulation until fully saturated, then dabbed on paper towels to dry to surface and the initial and final weight used to determine the amount of liquid which was adsorbed. This technique may not be feasible as a manufacturing process but nonetheless provided an additional assessment of adsorption potential.
Results indicated that Florite® R had the greatest potential to maximize drug potency as it achieved the highest loading level across all three techniques.

TABLE 10

| | Max Adsorbent | Max Tablet | Max Tablet |
|---|---|---|---|
| Adsorbent | Loading | Compression | Soaking |
| Syloid ® | 1.60x | 1.5x | 1.51x |
| XDP 3150 | (79.71% Etocas) | Force: 1.0 ton | Pure tablet: |
| | | (52.85% Etocas) | 105 mg, |
| | | | 3.9 mm |
| | | | (18.44% Etocas) |
| Neusilin ® | 2.50x | 1.75x | 0.71x |
| US2 | (79.71% Etocas) | Force: 0.75 ton | Pure tablet: |
| | | (52.85% Etocas) | 105 mg, |
| | | | 2.7 mm |
| | | | (18.44% Etocas) |
| Florite ® R | 4.00x | 2.25x | 1.83x |
| | (27.58% Etocas) | Force: 1.0 ton | Pure tablet: |
| | | (18.44% Etocas) | 50 mg, |
| | | | 2.2 mm |
| | | | (18.44% Etocas) |

Note:
values expressed as ratio (e.g., 1.60x = 1 part adsorbent + 1.60 parts lipid)

Single-layer tablets
   Florite® R was selected as the adsorbent based on the results of the adsorption assessment in previous section.
   Three (3) formulations containing ^{Florite}® R (2.25x) and various excipients were employed to form hard tablets. Friability was measured at the compression force which yielded the hardest tablets.

TABLE 11

| | | Compression | | Friability |
|---|---|---|---|---|
| Formu- | | Force | Hardness | (200 |
| lation | Excipients | (tons) | (Kp) | drops) |
| A | 5% Avicel ® PH-101 | 0.5 | 4.2 | 0.85% |
| | and 5% Starch 1500 ® | | | |
| B | 5% Avicel ® PH-101 | 0.5 | 3.9 | 4.74% |
| | and 5% Lactose Fast | | | |
| | Flo ® 316 | | | |
| C | 10% Polyethylene | 0.75 | 3.6 | 0.43% |
| | glycol (PEG) | | | |

Formula A:

| Material | Amount (g) | % w/w |
|---|---|---|
| SEDDS (9.562% w/w Rac potency) | 6.23 | 62.3 |
| Calcium silicate (Florite ® R) | 2.77 | 27.7 |
| Avicel ® PH-102 | 0.5 | 5.0 |
| Starch 1500 ® | 0.5 | 5.0 |
| TOTAL | 10.0 | 100.0 |

Tablet Weight: 840 mg
Dose: 50 mg racecadotril
Punch: 0.4062″ round concave
Force: 0.5 tons
Formula B:

| Material | Amount (g) | % w/w |
|---|---|---|
| SEDDS (9.562% w/w Rac potency) | 6.23 | 62.3 |
| Calcium silicate (Florite ® R) | 2.77 | 27.7 |
| Microcrystalline cellulose (Avicel ® PH-102) | 0.5 | 5.0 |
| Lactose monohydrate (Fast Flo ® 316) | 0.5 | 5.0 |
| TOTAL | 10.0 | 100.0 |

Tablet Weight: 840 mg
Dose: 50 mg racecadotril
Punch: 0.4062″ round concave
Force: 0.5 tons
Formula C:

| Material | Amount (g) | % w/w |
|---|---|---|
| SEDDS (9.562% w/w Rac potency) | 6.23 | 62.3 |
| Calcium silicate Florite ® R | 2.77 | 27.7 |
| Polyethylene glycol (PEG 4000) | 1.00 | 10.0 |
| TOTAL | 10.0 | 100.0 |

Tablet Weight: 840 mg
Dose: 50 mg racecadotril
Punch: 0.4062″ round concave
Force: 0.75 tons
Theoretical weights (50 mg dose) using Florite° R (2.25×) and 10% excipient are as follows:

| Formu- | Etocas ® 35 | Tablet Weight |
|---|---|---|
| lalation | (% w/w) | (mg) |
| 1 | 79.71 | 854 |
| 2 | 52.85 | 906 |
| 3 | 27.58 | 998 |
| 4 | 18.44 | 1,030 |
| 5 | 9.26 | 1,084 |

Compression-coated tablets
   This design allows the adsorbent to have a higher loading level because the coating layer prevents lipid from leaking out of the core upon compression.
   Tablets were successfully produced using Florite° R (3×) and various excipients for the coating layer.
   Core Formula

| Material | Amount (g) | % w/w |
|---|---|---|
| SEDDS (9.329% w/w Rac potency) | 3.96 | 75 |
| Calcium silicate (Florite ® R) | 1.32 | 25 |

Coating Layer A

| Material | Amount (g) | % w/w |
|---|---|---|
| Microcrystalline cellulose (Avicel ® PH-102) | 100 | 50 |
| Pregelatinized starch (Starch 1500 ®) | 100 | 50 |
| TOTAL | 10.0 | 100.0 |

Dose: 54 mg racecadotril
Core: 772 mg
Coating A: 550 mg
Total tablet: 1,322 mg
Core punch: 0.6875″×0.2812″ (0.25 tons)
Coating layer punch: 0.7500″×0.3750″×0.58″ (0.5 tons)
Coating Layer B

| Material | Amount (g) | % w/w |
|---|---|---|
| Microcrystalline cellulose (Avicel ® PH-102) | 100 | 50 |
| Lactose monohydrate (Fast Flo ® 316) | 100 | 50 |
| TOTAL | 10.0 | 100.0 |

Dose: 54 mg racecadotril
Core: 772 mg
Coating B: 550 mg
Total tablet: 1,322 mg
Core punch: 0.6875″×0.2812″ (0.25 tons)
Coating layer punch: 0.7500″×0.3750″×0.58″ (0.5 tons)
Theoretical Tablets weights (50 mg dose) using Florite® R (3×) are as follows:

| | Etocas ® 35 | Tablet Weight |
|---|---|---|
| Formula | (% w/w) | (mg) |
| 1 | 79.71 | 1,259 |
| 2 | 52.85 | 1,302 |
| 3 | 27.58 | 1,379 |
| 4 | 18.44 | 1,406 |
| 5 | 9.26 | 1,451 |

While the invention has been described above with reference to specific embodiments thereof, it is apparent that many changes, modifications, and variations can be made without departing from the inventive concept disclosed herein. Accordingly, it is intended to embrace all such changes, modifications, and variations that fall within the spirit and broad scope of the appended claims.

1. A method for treating a subject experiencing diarrhea, comprising administering to the subject a composition comprising racecadotril, at least one surfactant, and a lipid, wherein said racecadotril achieves a maximum plasma concentration (Cₘₐₓ) greater than about 300 ng/ml in said subject.

2. The method of claim 1, wherein the subject has been diagnosed with inflammatory bowel disease.

3. The method of claim 1, wherein the subject has been diagnosed with irritable bowel syndrome.

4. The method of claim 1, wherein a maximum plamsa concentratin (Cₘₐₓ) is achieved at about 1.5 to about 2.5 hours after ingestion.

5. The method of claim 1, wherein the racecadotril is maintained at a level above about 300 ng/ml for at least about 3.5 to about 5 hours after ingestion.

6. The method of claim 1, wherein the composition comprises an average droplet size selected from the group consisting of about 200 nm to about 15 nm, about 70 nm to about 20 nm, about 40 nm to about 20 nm, about 25 nm.

7. The method of claim 1, wherein the AUC v. reference ratio is selected from the group consisting of about 1.8 to about 3.5, about 2 to about 3.3, and about 3.3.

8. The method of claim 1, wherein the composition comprises about 8.0 wt. % to about 10.0 wt. % racecadotril, about 88 wt. % to about 91 wt. % surfactant, and about 1 wt. % to about 2 wt. % lipid, wherein each wt. % is based upon 100 ml of the composition.

9. The method of claim 1, wherein the composition comprises about 3.0 wt. % to about 7.0 wt. % racecadotril, about 40 wt. % to about 53 wt. % of surfactant in total, about 40 wt. % to about 53 wt. % lipid, wherein each wt. % is based upon 100 ml of the composition.
